(19) United States
(12) Patent Application Publication
(10) Pub. No.: US 2025/0232880 A1
(43) Pub. Date: Jul. 17, 2025

(54) FLEXIBLE TRANSDUCERS FOR BIOMETRIC DATA ACQUISITION

(71) Applicant: Organic Robotics Corporation, Ithaca, NY (US)

(72) Inventors: Robert Shepherd, Ithaca, NY (US); Ilayda Samilgil, Syracuse, NY (US); Kavinkumar Ramasamy Murthi, Irving, TX (US); Matthew Kirby, San Angelo, TX (US); Thomas Sipple, Ithaca, NY (US)

(21) Appl. No.: 18/849,930

(22) PCT Filed: Mar. 24, 2022
(86) PCT No.: PCT/US2022/021751

§ 371 Date: Sep. 23, 2024

(51) Int. Cl. G16H 50/30; G16H 40/63

(52) U.S. Cl. CPC G16H 50/30; G16H 40/63

(57) ABSTRACT

The following disclosure presents a system for high-speed biometric data acquisition comprising flexible optical fibers that may be anchored in a garment or wearable band. The flexible optical fibers are configured to collect biometric data, such as heartrate, a respiratory parameter, muscle contraction, and/or joint movement, at a sampling rate of at least 10 kilosamples per second, over a large dynamic range, and at high sensitivity and accuracy. The system further comprises a wearable modular electronics pod comprising a processor and transmitter for sending the biometric data to a wireless node. The present disclosure also presents a method for high-speed data acquisition comprising collecting biometric data at a sampling rate of at least 10 kilosamples per second and transmitting a wireless signal comprising the biometric data to a receiver at a speed of at least 0.1 kbps.

### FIELD OF THE DISCLOSURE

This disclosure relates to systems and methods for obtaining biometric data. Specifically, this disclosure relates to flexible transducers, such as flexible optical fibers, that may be used to acquire biometric data rapidly, over a large dynamic range, and at high sensitivity and accuracy.

### BACKGROUND

Humans move very fast, particularly in sports. For example, a baseball pitcher throws a ball at more than 90 mph; this high speed is a result of shoulder rotations that operate at kHz frequencies, sometimes more than 7 kHz. Signal processing requires oversampling for high quality measurements of signals; for sinusoidal waveforms, Nyquist criteria is ˜2 times oversampling. That would require 14 kilosamples per second (ksps) sampling rates for 7 kHz speeds, except that human motion is rarely sinusoidal. For the high speed, non-sinusoidal and aperiodic motions that humans perform, e.g. a fastball pitch, oversampling at ten times the rates is preferred. Therefore, to accurately measure a high speed motion like pitching, sampling at 70 kilosamples per second would be ideal. Measurement of biometric motion at these rates, however, is currently not possible with conventional technology.

Accordingly, there is a need for systems and methods that can acquire biometric data using flexible transducers, such as flexible optical fibers, rapidly, over a large dynamic range, and at high sensitivity and accuracy.

### SUMMARY

The following description presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later.

In some embodiments, the present disclosure provides a system for high-speed biometric data, including stretchable fiber optic wearables. For instance, in some embodiments, the system includes a first flexible transducer, where the first flexible transducer is configured to be worn around or along a portion of a person's body and configured to obtain biometric data indicating a movement of the person's body, a process, and a transmitter. In some embodiments, the system is configured to collect biometric data at a sampling rate of at least 10 kilosamples per second (ksps). In some embodiments, the system is configured to collect biometric data at a sampling rate of at least 100 kilosamples per second. In some embodiments, the system is configured to collect biometric data at a sampling rate of at least 200 kilosamples per second. In some embodiments, the biometric data comprises at least one of heartrate, chest excursion, tidal volume, minute volume, vital capacity, breathing rate, joint angle or position, joint velocity, joint acceleration, jerk, snap, joint stiffness, muscle activity, and muscle fatigue. In some embodiments, the biometric data indicating a movement of a person's body are measured at kHz frequencies.

In some embodiments, the first flexible transducer comprises an optical fiber, wherein the optical fiber comprises a first end configured to receive light emitted by a light source and a second end configured to transmit light to a detector. In some embodiments at least a portion of the optical fiber is deformable and has a propagation loss parameter configured to increase when the deformable section is deformed. In some embodiments, a first end and a second end of the first flexible transducer are anchored in a wearable garment. In some embodiments, a first end and a second end of the first flexible transducer are anchored in a wearable band. In some embodiments, the wearable band is a horizontal or transverse band configured to be worn across a user's chest or torso, around a wrist or ankle, or around a user's muscle. For example, the wearable band may be worn transversely across a muscle, such that the wearable band wraps circumferentially around a user's arm muscle (e.g., bicep) or leg muscle (e.g., quadriceps, hamstrings, calf muscles, etc.). In some embodiments, the wearable band is an orthogonal band configured to be worn along a user's joint.

In some embodiments, the system further comprises a second flexible transducer, wherein the first flexible transducer is worn in a first area of the person's body having a first rate of motion and the second flexible transducer is worn in a second area of the body having a second rate of motion. In some embodiments, the second rate of motion is greater than the first rate of motion, and at least one of a data acquisition rate and a data transmission rate of the second flexible transducer is greater than a corresponding rate of the first flexible transducer.

In some embodiments, the system further comprises an electronics pod configured to house at least one of the light source, detector, processor, and transmitter. In some embodiments, the electronics pod is capable of being coupled to and decoupled from the wearable garment or wearable band.

In some embodiments, the present disclosure provides a method for high-speed data acquisition comprising collecting biometric data at a sampling rate of at least 10 kilosamples per second and transmitting a wireless signal comprising the biometric data to a receiver at a speed of at least 0.1 kbps. In some embodiments, the data is transmitted at a speed of at least 10 kbps. In some embodiments, the step of collecting the biometric data is performed using the system described herein.

In some embodiments, the present disclosure provides a method for high-speed data acquisition comprising disposing an extensible transducer around a portion of a person's chest, the extensible transducer being configured to extend in response to movement of the person's chest, and, based on movements detected by the transducer at the person's chest, simultaneously measuring a heartrate and at least one respiration parameter of the person using the extensible transducer. In some embodiments, the extensible transducer comprises an optical fiber, wherein the optical fiber comprises a first end configured to receive light emitted by a light source and a second end configured to transmit light to a detector. In some embodiments, at least a portion of the optical fiber is deformable and has a propagation loss parameter configured to increase when the deformable section is deformed

In some embodiments, the extensible transducer is configured to obtain data indicating the movement of the person's chest over a dynamic range from an extension of about 0% to an extension of about at least 15%. In some embodiments, the extensible transducer is configured to obtain data indicating the movement of the person's chest to an extension of about at least 25%. In some embodiments, the extensible transducer is configured to obtain data indicating the movement of the person's chest to an extension of about at least 50%. In some embodiments, over the dynamic range, the extensible transducer is configured to measure the movement of the person's chest to within one millimeter. In some embodiments, over the dynamic range, the extensible transducer is configured to measure the movement of the person's chest to within one- tenth of a millimeter. In some embodiments, the extensible transducer is configured to measure forces less than 1 N, and wherein the force is measureable to within 0.01 N. In some embodiments, the extensible transducer is configured to simultaneously measure both a heartrate and a respiration parameter of the person. In some embodiments, the extensible transducer is configured to measure strain in the range of about 100 μm to about 10 cm, wherein the strain is measurable to within 100 μm strain. In some embodiments, the biometric data is collected at a sampling rate of at least 100 kilosamples per second. In some embodiments, the biometric data is collected at a sampling rate of at least 200 kilosamples per second. In some embodiments, the biometric data indicating a movement of a person's body are measured at kHz frequencies.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1A shows a schematic of an exemplary flexible transducer having one or more fiber sections, according to an embodiment of the present disclosure.

FIG. 1B shows a schematic of an exemplary flexible transducer having a single fiber section, according to an embodiment of the present disclosure.

FIG. 2 shows an exemplary system for high-speed biometric data acquisition, wherein the flexible transducer is in an orthogonal band configuration, according to an embodiment of the present disclosure.

FIG. 3 shows an exemplary system for high-speed biometric data acquisition, wherein the flexible transducer is in a horizontal or transverse band configuration, according to an embodiment of the present disclosure.

FIG. 4A shows an exemplary alternative configuration of the system for high-speed biometric data acquisition of FIG. 3, wherein the light source, detector, processor, and transmitter are housed together within an electronics pod, according to an embodiment of the present disclosure.

FIG. 4B shows exemplary biometric data obtained from a system for high-speed biometric data acquisition, according to an embodiment of the disclosure.

FIGS. 5A-5B show an exemplary system for high-speed biometric data acquisition, comprising a plurality of flexible transducers is in a wearable garment configuration, according to an embodiment of the present disclosure.

FIG. 5C shows a close-up view of a section of the garment of FIG. 5A with an integrated optical fiber, according to an embodiment of the present disclosure.

FIG. 6 shows an exemplary system for high-speed biometric data acquisition, comprising a plurality of flexible transducers is in a wearable garment configuration, according to an embodiment of the present disclosure.

FIGS. 7A-7E show an exemplary electronics pod, according to an embodiment of the disclosure.

FIG. 8 shows an exemplary method of using the system for high-speed biometric data acquisition, according to an embodiment of the present disclosure.

FIG. 9 shows a flowchart detailing an exemplary method for biometric data acquisition, according to an embodiment of the present disclosure.

FIG. 10 shows a flowchart detailing an exemplary method for biometric data acquisition, according to an embodiment of the present disclosure.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

Unless defined otherwise, all terms of art, notations and other technical terms or terminology used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

Unless otherwise indicated or the context suggests otherwise, as used herein, “a” or “an” means “at least one” or “one or more.”

This description may use relative spatial and/or orientation terms in describing the position and/or orientation of a component, apparatus, location, feature, or a portion thereof. Unless specifically stated, or otherwise dictated by the context of the description, such terms, including, without limitation, top, bottom, above, below, under, on top of, upper, lower, left of, right of, in front of, behind, next to, adjacent, between, horizontal, vertical, diagonal, longitudinal, transverse, radial, axial, etc., are used for convenience in referring to such component, apparatus, location, feature, or a portion thereof in the drawings and are not intended to be limiting.

Furthermore, unless otherwise stated, any specific dimensions mentioned in this description are merely representative of an exemplary implementation of a device embodying aspects of the disclosure and are not intended to be limiting.

To the extent used herein, the term “adjacent” refers to being near or adjoining. Adjacent objects can be spaced apart from one another or can be in actual or direct contact with one another. In some instances, adjacent objects can be coupled to one another or can be formed integrally with one another.

To the extent used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the embodiments described herein.

To the extent used herein, the terms “optional” and “optionally” mean that the subsequently described, component, structure, element, event, circumstance, characteristic, property, etc. may or may not be included or occur and that the description includes instances where the component, structure, element, event, circumstance, characteristic, property, etc. is included or occurs and instances in which it is not or does not.

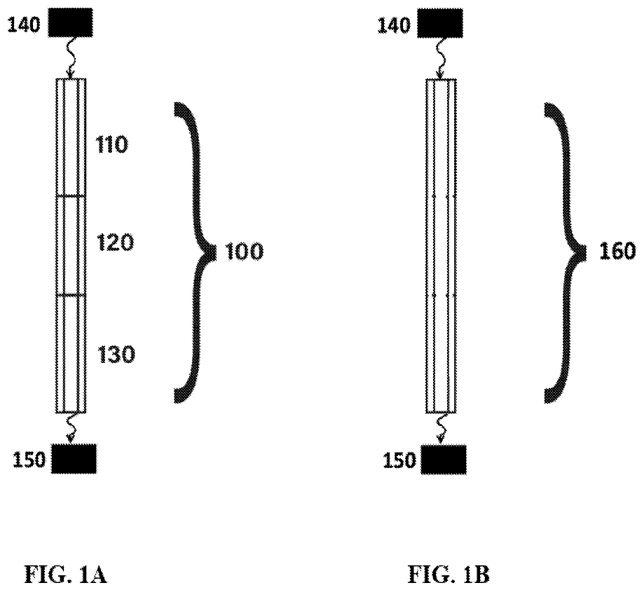

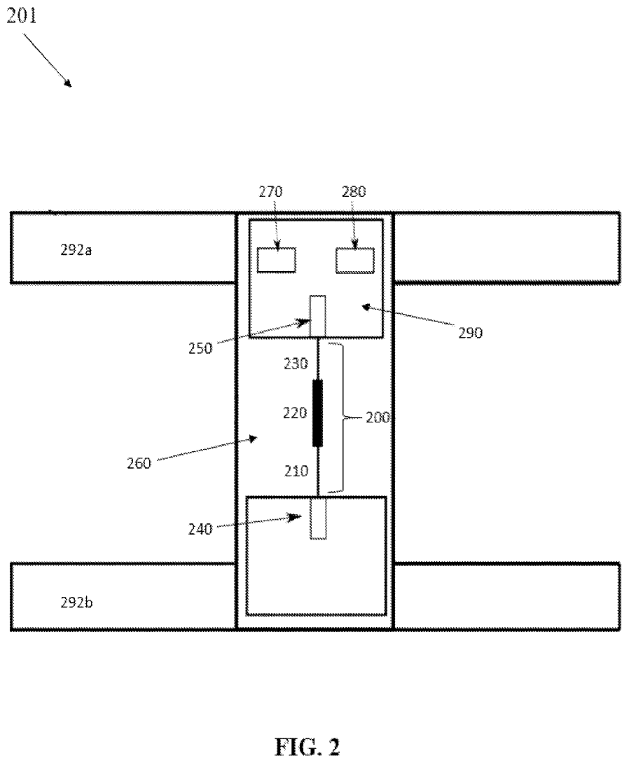

FIG. 1A shows an exemplary flexible transducer 100, according to an embodiment of the present disclosure. For purposes of this disclosure, the meaning of the term “flexible” is not particularly limited. For instance, in some embodiments, the flexible transducer 100 may be a transducer that is malleable, pliable, extensible, elastic, deformable, and/or capable of bending or flexing in accordance with a person's body movements. In some embodiments, the flexible transducer 100 is an optical fiber. In some embodiments, the transducer 100 may include one or more of a first fiber section 110, a second fiber section 120, and a third fiber section 130. The transducer 100 may further include a light source 140 and a detector 150. Although three fiber sections are shown in this exemplary embodiment, any number of fiber sections may be used. In some embodiments, the first fiber section 110 and third fiber section 130 may be low-loss fiber sections. For example, the first and/or third fiber sections may be composed of transparent plastic core of poly(methyl methacrylate). In other embodiments, the first and/or third fiber sections may be composed on glass. The core of the first and/or third fiber sections may have an optical attenuation coefficient of less than 0.1 dB cm⁻¹, less than 1 dB m⁻¹, or less than 0.1 dB m⁻¹. As used herein, the term optical attenuation coefficient is used to refer to optical loss per unit length. The term propagation loss parameter refers to the amount of optical loss over an entire length of a fiber. Specifically, as the fiber bends, light leaves the fiber. Yet as the fiber is stretched and extended, the light travels through more material. Therefore, for the same absorption coefficient, the loss of light increases with deformation. An index of refraction of the core of the first and/or third fiber sections may be approximately 1.5. In some embodiments, the first and/or third fiber sections may have a cladding, or coating. In some embodiments, the cladding may be made, in whole or in part, of Teflon. In some embodiments, the cladding may have an index of refraction approximately 1.4. In some embodiments, the cladding provides resistance to compressive strain and tensile deformation, thus maintaining the structural integrity of the fiber section. In some embodiments, the first fiber section 110 may have a length that is equal to or greater than 1 cm, 10 cm, 1 m, or 10 m. In some embodiments, the third fiber section 130 may have a length that is equal to or greater than 1 cm, 10 cm, 1 m, or 10 m.

In some embodiments, the second fiber section 120 may be an extensible fiber section in which a propagation loss parameter varies as the second fiber section 120 is stretched. For example, the second fiber section 120 may have an ultimate elongation of at least 5%, 10%, 20%, 50%, 75%, 100%, 150%, 200%, 300%, or 500%. In some embodiments, a propagation loss parameter may increase as the second fiber is stretched. For example, an optical attenuation coefficient of the second fiber section 120 may be substantially constant, such that as a length of the second fiber section 120 increases, a total amount of light loss over the length of the second fiber section 120 may increase.

In some embodiments, the second fiber section 120 may be composed of transparent elastomer core such as poly(urethane). The second fiber section 120 may have an index of refraction approximately 1.5. The second fiber section 120 may have an optical attenuation coefficient of approximately 0.01, 0.05, 0.1, 0.5, 1, 10, 100, 1000 dB cm⁻¹. In some embodiments, the second fiber section 120 may include a cladding, or coating. For example, the cladding may be made of an elastomer or plastic of lower index of refraction than the core. Silicone (having an index of refraction approximately 1.4), Teflon (having an index of refraction of approximately 1.4) are examples of suitable cladding materials. In some embodiments, the second fiber section 120 may not include a cladding. For example, the second fiber section 120 may be surrounded by air, which has an index of refraction of approximately 1.0. In some embodiments, the second fiber section 120 may be a waveguide having any of the properties, or made according to any of the methods, described in U.S. Patent Publication No. 2019/0056248. In some embodiments the second fiber section 120 may have a length that is greater than 0.05 cm, 0.1 cm, 0.5 cm, 1 cm, 2 cm, or 3 cm. In some embodiments, the second fiber section 120 may have a length that is less than 5 cm, 10 cm, 20 cm, 50 cm, or 100 cm.

In some embodiments, the light source 140 may be a light-emitting diode. For example, a photodiode or laser diode may be used. In some embodiments, the light source 140 may have a peak wavelength that is between 400 nm and 1 mm. In some embodiments, the detector 150 may be a phototransistor, photodiode, or complementary metal-oxide-semiconductor (CMOS). The fiber may have a first end that is configured to receive light emitted by the light source 140. For example, the light source 140 may be, e.g., attached to, disposed adjacent to, or embedded in whole or in part within the first end of the fiber, such that light emitted by the light source 140 may enter and pass through the core of the fiber. The detector 150 may be arranged at a second end of the fiber, opposite the first, to receive light that travels through the fiber. For example, the detector 150 may be, e.g., attached to, disposed adjacent to, or embedded in whole or in part within the second end of the fiber, such that light that passes through the fiber may reach and be detected by the detector 150.

In some embodiments, the second fiber section 120 may be bonded to the first fiber section 110 such that light may pass from the first fiber section 110 to the second fiber section 120. In embodiments that include an optional third fiber section 130, the third fiber section 130 may be bonded to the second fiber section 120 such that light may pass from the second fiber section 120 to the third fiber section 130. Thus, the fiber may be arranged such that when the first end is coupled to a light source 140 and the second end is coupled, directly or indirectly (e.g., via an optional third fiber section) to a detector 150, light travels from the light source 140, through the first fiber section 110, the second fiber section 120, and the optional third fiber section 130 and to the detector 150.

As shown in FIG. 1B, the exemplary flexible transducer 100 may alternatively include only a single fiber section 160. The single fiber section 160 may be an extensible fiber section as described above with respect to FIG. 1A, and it may be made from the same materials, have the same cladding, and have the same or similar properties as described above with respect to second fiber section 120. For instance, the entire length of the single fiber section 160 of the flexible transducer 100 may be deformable. Thus, in some embodiments, as shown in FIG. 1B, the flexible transducer 100 does not include any non-extensible fiber sections. In some embodiments, single fiber section 160 has an optical attenuation coefficient in the range of about 0.01 dB/cm to about 1000 dB/cm.

FIG. 2 shows an exemplary system 201 for high-speed biometric data acquisition, according to an embodiment of the present disclosure. The system 201 comprises a flexible transducer 200, wherein the flexible transducer 200 has the same features and characteristics as those described above with respect to the flexible transducer 100. In some embodiments, the flexible transducer 200 comprises a first fiber section 210, a second fiber section 220, and a third fiber section 230, which have the same features and characteristics as those described above with respect to fiber sections 110, 120, and 130, respectively. For instance, in some embodiments, the first fiber section 210 and third fiber section 230 are inextensible, wherein the second fiber section 220 is extensible and can be deformed, extended, or contracted. The system 201 further comprises a light source 240 and detector 250, which correspond to the light source 140 and detector 140 described above.

In some embodiments, as shown in FIG. 2, the flexible transducer 200 may be positioned on, anchored in, or embedded within, a base 260. The base 260 may comprise a flexible band, a section of fabric, or a piece of a wearable garment. For instance, in a non- limiting example, base 260 may comprise a portion of a shirt, athletic top, sports bra, shorts, pants, leggings, spandex, spanx, leotard, unitard, singlet, swimsuit, compression sleeve, or other athletic gear worn against the body of a user. In some embodiments, the flexible transducer 200 may be attached to or embedded within the base 260 via stitching, embroidering, a mechanical fastener, an adhesive, or the like.

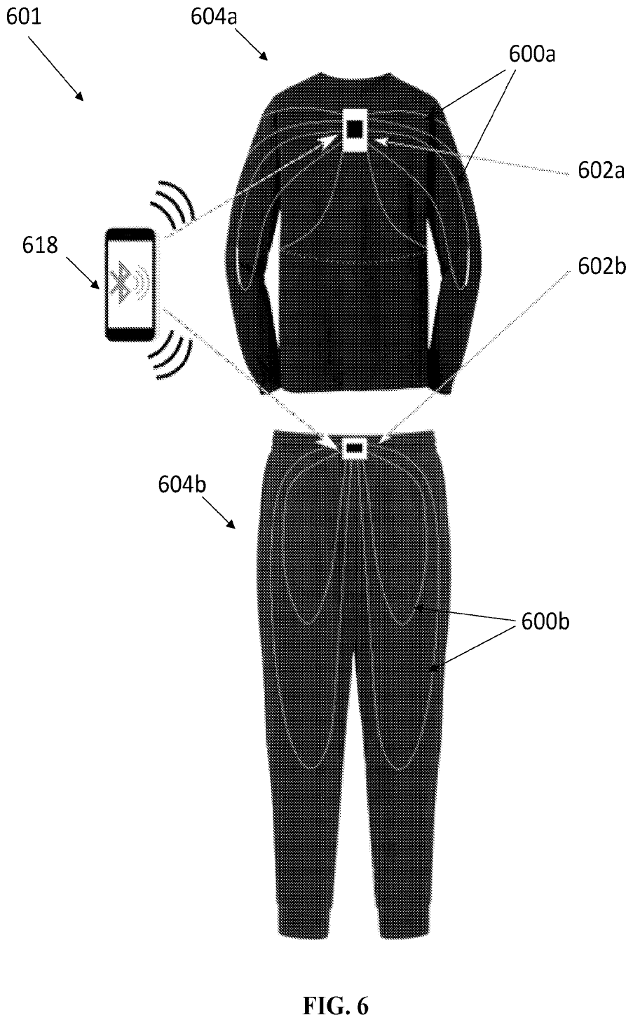

The system 201 for high-speed data acquisition may further comprise an electronics board 290, wherein the electronics board 290 comprises a processor 270 and a transmitter 280. Thus, in some embodiments, when the light source 240 emits an optical signal (not shown) that passes through the flexible transducer 200 and is received by the detector 250, processor 270 may process the detected signal and transmitter 280 may transmit the data related to the detected signal to a wireless node (shown in FIG. 6). In some embodiments, the processor 270 may comprise a microcontroller. In some embodiments, the processor 270 comprises an algorithm for isolating the desired biometric data signals and cancelling, or otherwise minimizing, signal noise. In some embodiments, the transmitter 280 may comprise a BLE transmitter that communicates data to a wireless node, such as a mobile phone, smart phone, computer, pager, tablet, laptop, etc. In some embodiments, the electronics board 290 comprising the processor 270 and transmitter 280 are housed within an electronics pod. In some embodiments, the electronics board 290 comprises additional electronic components, such as an analog-to-digital converter.

In some embodiments, as shown in FIG. 2, the base 260 comprising the flexible transducer 200, light source 240, detector 250, electronics board 290, processor 270, and transmitter 280 may be a flexible orthogonal band attached to one or more straps 292a, 292b, wherein the orthogonal band may be configured to span a joint on a user's body. For instance, in a non-limiting example, a first strap 292a may be positioned above a user's elbow, so as to be wrapped around a user's bicep, and a second strap 292a may be positioned below the user's elbow, so as to be wrapped around a user's forearm. In this example, in the orthogonal configuration, the length of the flexible transducer 200 is positioned along a user's elbow, and may be used to measure kinematic motions upon bending and straightening of the elbow, wherein the extensible portion of the flexible transducer 200 may be flexed and deformed in accordance with the elbow movement. In some embodiments, the flexible transducer may be used to measure biometric data, wherein the biometric data comprises at least one of joint angle or position, joint velocity, joint acceleration, jerk, snap, and joint stiffness. In some embodiments, joint position is measured in signal magnitude. In some embodiments, joint velocity is measured in signal change per second. In some embodiments, joint acceleration is measured in velocity per second. In some embodiments, joint jerk is a third derivative of position, measured in acceleration per second. In some embodiments, joint snap is the rate of change of jerk, measured in jerk per second. This biometric data may be used to capture and analyze high-speed bodily movements and motions for improved consistency and enhanced athletic performance.

In other examples, the one or more straps 292a, 292b may use to secure the flexible transducer 200 around other joints, such as the knee, shoulder, or ankle. In some embodiments, the one or more straps 292a, 292b may comprise a mechanical fastener (not shown) to fasten the ends of each strap 292a, 292b around a user's body part. The mechanical fasteners may comprise, for instance, at least one of hook-and-loop fasteners, male and female connectors, zippers, lip and tape fasteners, rivets and eyelets, cufflinks, buttons, snaps, clasps, clips, eyelets and lace, and safety pins. In other embodiments, the one or more straps 292a, 292b are integrated into a wearable garment such that no mechanical fasteners are necessary.

In some embodiments, the system 201 comprises a single flexible transducer 200. In other embodiments, the system 201 comprises at least one or more flexible transducers 200. For instance, in some embodiments, the system 201 comprises a plurality of flexible transducers 200.

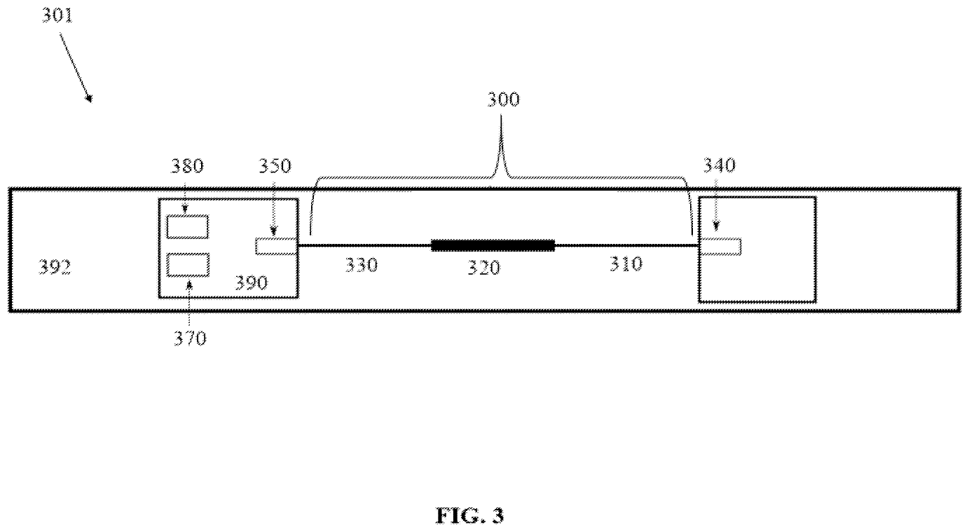

FIG. 3 shows an exemplary system 301 for high-speed biometric data acquisition, wherein the flexible transducer 300 is the horizontal or transverse configuration, according to an embodiment of the present disclosure. The system 301 comprises flexible transducer 300, wherein the flexible transducer 300 comprises a first fiber section 310, a second fiber section 320, and a third fiber section 330, light source 340, detector 350, electronics board 390, processor 370, transmitter 380, and base 392, each of which have the same features and characteristics as those corresponding elements described above. In some embodiments, base 392 is a flexible band configured to be worn, for instance, across a user's chest or torso, around a wrist or ankle, or around a user's muscle. For example, the flexible band may be worn transversely across a muscle, such that the flexible band wraps circumferentially around a user's arm muscle (e.g., bicep) or leg muscle (e.g., quadriceps, hamstrings, calf muscles, etc.). In this horizontal or transverse configuration, the system 301 is configured to measure biometric data, wherein the biometric data comprises at least one of heartrate, respiration parameter, muscle activity (e.g., contraction and relaxation), and muscle fatigue. Heartrate may be measured from a pulse beating, and may be measured in units of beats per minute. In some embodiments, the respiration parameter is at least one of more of chest excursion, tidal volume, minute volume, vital capacity, and breathing rate. In some embodiments, the system 301 may be configured to measure at least two different types of biometric data simultaneously, from a single flexible transducer 300 or from a plurality of flexible transducers 300. For instance, in some embodiments, a single flexible transducer 300 is configured to measure heartrate and a respiratory parameter simultaneously. In some embodiments, the system 301 is configured to collect biometric data at a sampling rate of at least 100 kilosamples per second.

In some embodiments, the flexible band 392 is configured to be worn around a person's chest, wherein the flexible transducer 300 is configured to extend in response to a movement of the person's chest. In some embodiments, the system 301 is configured to obtain data indicating the movement of the person's chest over a dynamic range, wherein the dynamic range comprises an extension of about 0% to an extension of about 15%. In some embodiments, the system 301 is configured to obtain data indicating the movement of the person's chest to an extension of about at least 25%. In some embodiments, the system 301 is configured to obtain data indicating the movement of the person's chest to an extension of about at least 50%. In some embodiments, the system 301 is configured to measure the movement of the person's chest to within one millimeter. In some embodiments, the system 301 is configured to measure the movement of the person's chest to within one-tenth of a millimeter. In some embodiments, the system 301 is configured to measure forces less than 1 N, and wherein the force is measureable to within 0.01 N. In some embodiments, the system 301 is configured to simultaneously measure both a heartrate and a respiration parameter of the person. In some embodiments, the system 301 is configured to measure strain in the range of about 100 μm to about 10 cm, wherein the strain is measurable to within 100 μm strain.

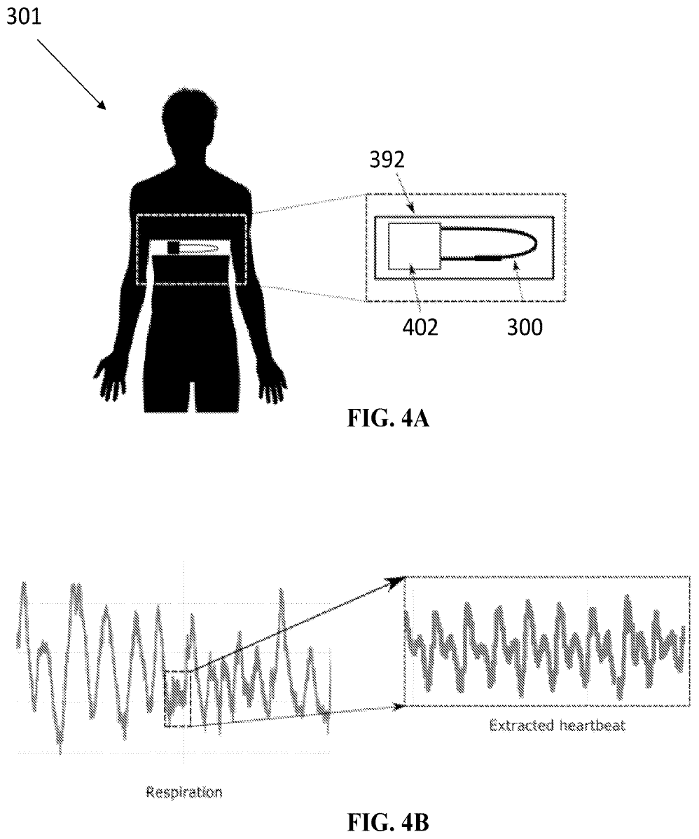

The arrangement and positioning of the light source 340 and detector 350 in relation to the electronic components, such as the processor 370 and transmitter 380, is not particularly limited. For instance, in some embodiments, the light source 340 may be positioned on one end of the band 392 and the detector 350, processor 370, and transmitter 380 may be positioned on the other end of the band 392 (as shown in FIG. 3). In an alternative embodiments, the light source 340, detector 350, processor 370, and transmitter 380 may all be housed together within an electronics pod 402 (as shown in FIG. 4A). In this alternative configuration, both ends of the flexible transducer 300 are connected to the electronics pod 402, such that the flexible transducer has a substantially looped shape, as opposed to linear shape.

FIG. 4B shows exemplary biometric data obtained from a system for high-speed biometric data acquisition, according to an embodiment of the disclosure. For instance, where a single flexible transducer is configured to measure both heartrate and a respiratory parameter simultaneously, the biometric data may comprise raw data showing a respiratory parameter and heartbeat data extracted from the respiratory data.

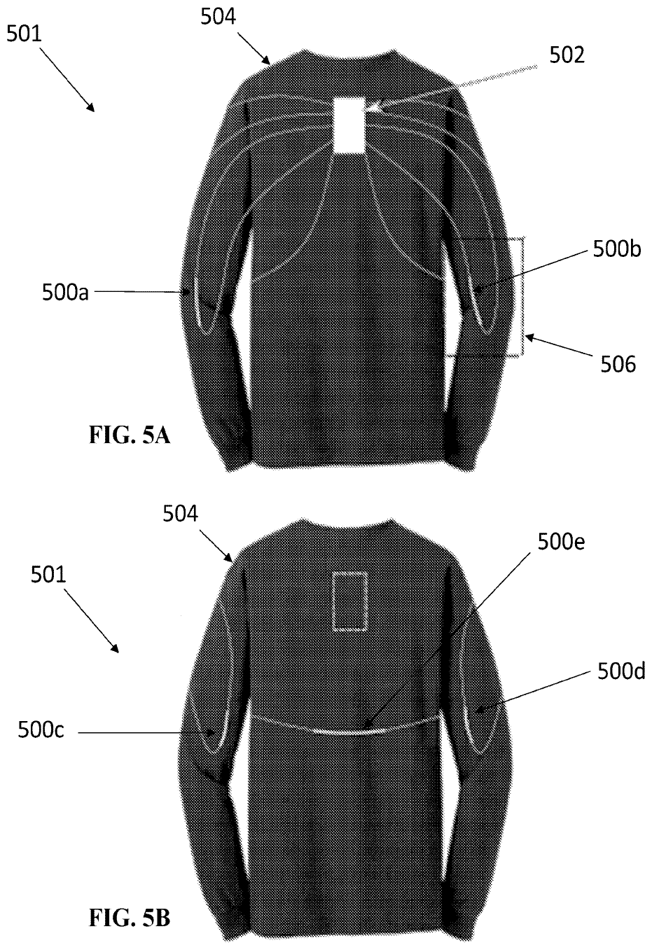

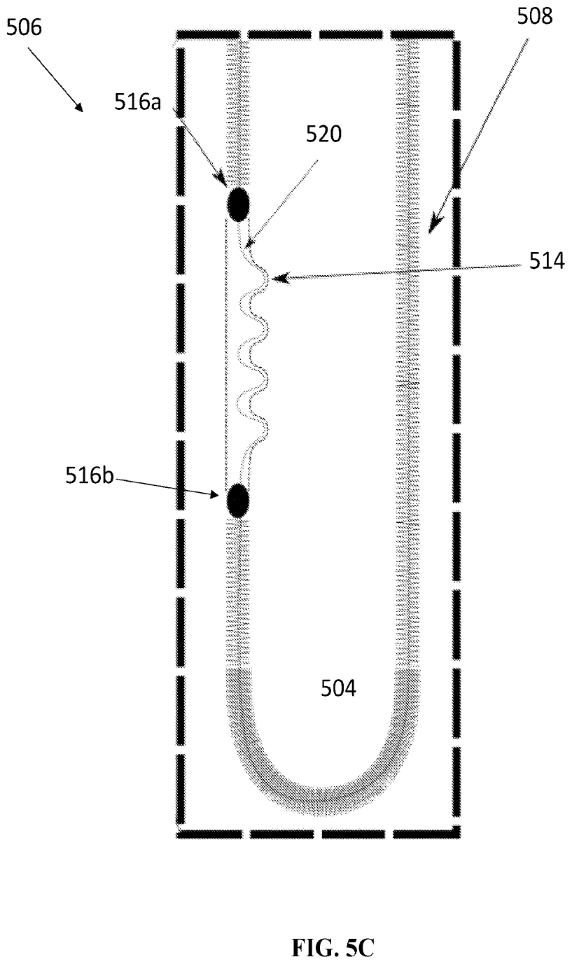

FIGS. 5A-5B show an exemplary system 501 for high-speed biometric data acquisition, comprising a plurality of flexible transducers 500a, 500b, 500c, 500d, 500e in a wearable garment configuration, according to an embodiment of the present disclosure. Each of the plurality of flexible transducers 500a, 500b, 500c, 500d, 500e may have the same features and characteristics as the corresponding flexible transducers 100, 200, 300 described above. FIG. 5A shows a front view of a garment 504 configured to be worn by a user, whereas FIG. 5B shows a back view. Although the garment 504 is depicted here as a long-sleeve shirt, the type of garment is not particularly limited for purposes of this disclosure. For instance, in some embodiments, the garment 504 may comprise a short-sleeve shirt, athletic top, sports bra, shorts, pants, leggings, spandex, spanx, leotard, unitard, singlet, swimsuit, compression sleeve, or other athletic gear worn against the body of a user.

In some embodiments, the plurality of flexible transducers 500a, 500b, 500c, 500d, 500e are configured to be positioned against select portions of the user's body for high-speed biometric data acquisition. For instance, the arrangement of the plurality of flexible transducers 500a, 500b, 500c, 500d, 500e within or on the garment 504 may be configured so as to collect and monitor biometric data from at least one of the user's chest, shoulders, back muscles, core, and arm muscles (e.g., biceps and triceps) upon movement by the user. As described above, the biometric data comprises at least one of heartrate, respiration parameter, muscle activity (e.g., contraction and relaxation), and muscle fatigue. Both ends of each of the plurality of flexible transducers 500a, 500b, 500c, 500d, 500e are connected to electronics pod 502 (entering it through multiple locations), which also houses the light source, detector, processor, and transmitter.

The means for securing of the plurality of flexible transducers 500a, 500b, 500c, 500d, 500e within or on the garment 504 is not particularly limited for purposes of this disclosure. For instance, the transducers 500 may be secured on the outside surface of the garment 504, the inside surface of the garment 504, or embedded within the garment 504. In a preferred embodiment, the transducers 500 are embedded within the garment 504, so as to minimize any potential chafing or irritation for the user during movement. In some embodiments, the transducers 500 may be secured to the garment via at least one of stitching, embroidering, a mechanical fastener, an adhesive, and the like.

FIG. 5C shows a close-up view of section 506 of the garment 504 of FIG. 5A with an integrated optical fiber 500b, according to an embodiment of the present disclosure. The close-up view shows an embroidered channel 508 for guiding the fiber 500b through the garment 504 so as to position the fiber 500b against a selected portion of the user's body for high-speed biometric data acquisition. An extensible section 520 of the fiber 500b forms a wave-like shape within outline stitching 514. Upon movement by a user wearing the garment 504, the extensible section 520 may stretch out and expand, and then contract back into place as biometric data is collected. The fiber 500b may be adhered to the garment 504 via at least one attachment point 516a, 516b, such as with an adhesive.

FIG. 6 shows an exemplary system 601 for high-speed biometric data acquisition, comprising a plurality of flexible transducers is in a wearable garment configuration, according to an embodiment of the present disclosure. In some embodiments, system 601 comprises one or more garments 604a, 604b, wherein each garment 604a, 604b comprises one or more flexible transducers 600a, 600b for high-speed biometric data acquisition and an electronics pod 602a, 602b for processing and transmitting the data to a wireless node 618. The number of flexible transducers is not particularly limited for purposes of this disclosure. In some embodiments, at least one garment 604a may comprise a shirt and at least one garment 604b may comprise pants for measuring biometric data at various parts of a user's body when wearing the garments 604a, 604b. In some embodiments wireless node 618 may comprise at least one of a radio frequency communication device, mobile phone, smart phone, computer, pager, tablet, and a laptop. In some embodiments, the radio frequency communication device is a Bluetooth-enabled device. In some embodiments (not shown), the biometric data is transmitted to a wired node via a wired connection, instead of wireless node 618.

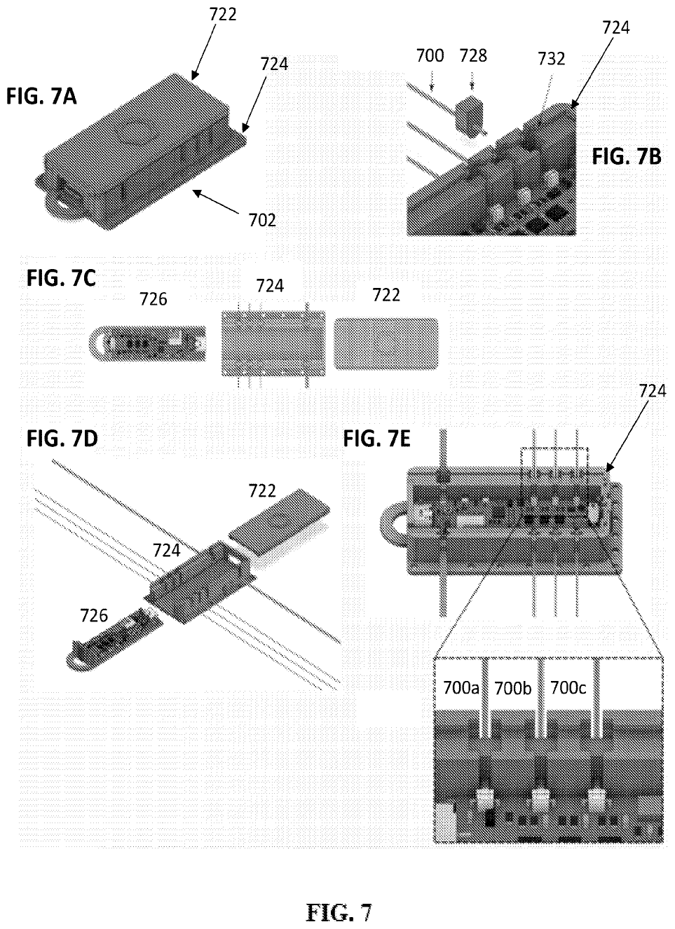

FIGS. 7A-7E show an exemplary electronics pod 702, according to an embodiment of the present disclosure. In some embodiments, the electronics pod 702 is configured to house at least one of the light source, detector, processor, and transmitter. In some embodiments, the electronics pod 702 is configured to secure the ends of at least one flexible transducer 700. In some embodiments, the electronics pod 702 is configured to secure the ends of a plurality of flexible transducers 700 to allow for the collection of multiple biometric data simultaneously. In some embodiments, the electronics pod 702 is capable of being coupled to and decoupled from a wearable garment or wearable band to, for instance, allow for washing of the garment or wearable band and/or move the modular electronics pod 702 into another garment or wearable band.

FIG. 7A shows the exterior of the electronics pod 702, comprising a removable cover 722 and an enclosure base 724. As shown in FIG. 7B (and close-up view in FIG. 7E), at least one flexible transducer 700a, 700b, 700c may be secured to the enclosure base 724 of the electronics pod 702 via a tip 728 attached to one end of the transducer 700. The tip 728 may be configured to fit within opening 732, so as to secure the transducer 700 in place. In some embodiments, the at least one transducer 700a, 700b, 700c may be secured so as to align with the light source and detector, which are also housed within the electronics pod 702. FIG. 7C shows the components of the electronics pod 702 in a top-down exploded view, showing the removable cover 722, enclosure base 724, and electronics drawer 726, whereas FIG. 7D shows an exploded isometric view of the same. In some embodiments, the electronics drawer 726 comprises a removable electronics board, wherein the electronics board is a printed circuit board (PCB) providing the processor and transmitter thereon. In some embodiments, the electronics pod 702 may be secured to a garment via the enclosure base 724.

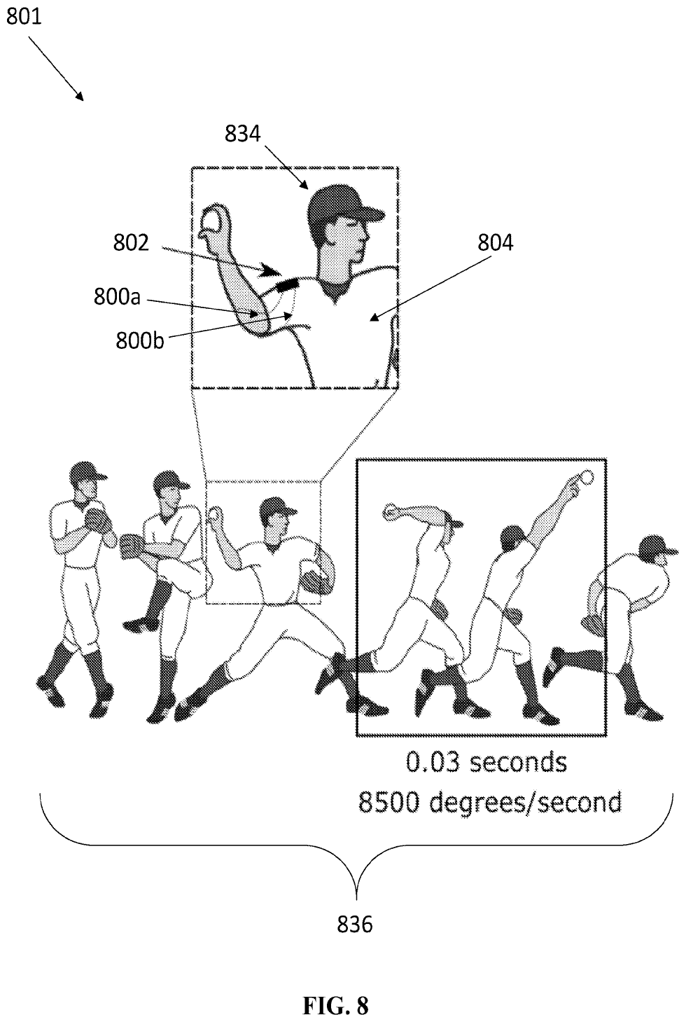

FIG. 8 shows an exemplary method of using a system 801 for high-speed biometric data acquisition, according to an embodiment of the present disclosure. As a non-limiting example, a baseball pitcher 834 throws a pitch while wearing a garment 804 comprising integrated flexible transducers 800a, 800b connected to an electronics pod 802. The biometric data collected as the baseball pitcher 834 goes through the motion 836 of throwing the pitch is captured by the system 801 and sent to a wireless node through the transmitter. The biometric data collected from the sensing system 801 on the baseball pitcher's shoulder may be used to determine, for instance, a moment of highest velocity and maximum arm rotational speed. In other examples, the biometric data collected from sensing system 801 may be used to determine arm and/or shoulder acceleration, snap, or jerk that can be used for improving pitching speed or preventing injuries.

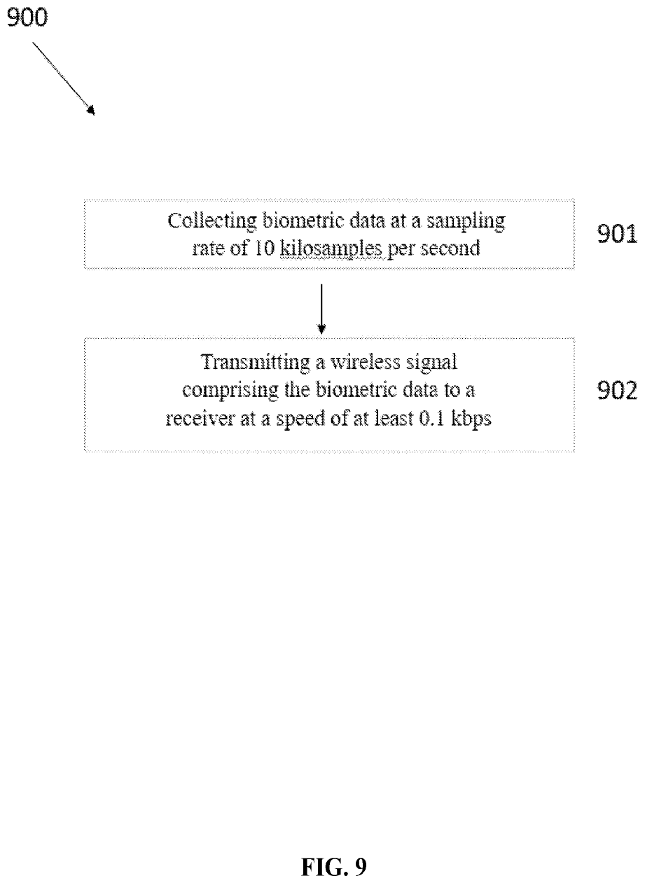

FIG. 9 shows a flowchart detailing an exemplary method 900 for biometric data acquisition, according to an embodiment of the present disclosure. The method comprises collecting biometric data at a sampling rate of at least 10 kilosamples per second (901) and transmitting a wireless signal comprising the biometric data to a receiver at a speed of at least 0.1 kbps (902). In some embodiments, the step of collecting the biometric data is performed using a system comprising (i) a first flexible transducer configured to be worn around or along a portion of a person's body, wherein the first flexible transceiver is configured to obtain biometric data indicating a movement of the person's body, (ii) a microcontroller, (iii) an analog-to-digital converter, and (iv) a transmitter. In some embodiments, the biometric data is collected at a sampling rate of at least 100 kilosamples per second. In some embodiments, the biometric data is collected at a sampling rate of at least 200 kilosamples per second. In some embodiments, the first flexible transducer is coupled to a modular electronics pod capable of being coupled to and decoupled from a wearable garment or wearable band. In some embodiments, the biometric data indicating a movement of a person's body are measured at kHz frequencies.

In some embodiments, the first flexible transducer comprises an optical fiber, wherein the optical fiber comprises a first end configured to receive light emitted by a light source and a second end configured to transmit light to a detector, wherein at least a portion of the optical fiber is deformable and has a propagation loss parameter configured to increase when the deformable section is deformed. In some embodiments, a first end and a second end of the first flexible transducer are anchored in a wearable garment. In some embodiments, a first end and a second end of the first flexible transducer are anchored in a wearable band. In some embodiments, the wearable band is a horizontal or transverse band configured to be worn across a user's chest or torso, around a wrist or ankle, or around a user's muscle. For example, the wearable band may be worn transversely across a muscle, such that the wearable band wraps circumferentially around a user's arm muscle (e.g., bicep) or leg muscle (e.g., quadriceps, hamstrings, calf muscles, etc.). In some embodiments, the wearable band is an orthogonal band configured to be worn along a user's joint.

In some embodiments, the biometric data comprises at least one of heartrate, chest excursion, tidal volume, minute volume, vital capacity, breathing rate, joint angle or position, joint velocity, joint acceleration, jerk, snap, joint stiffness, muscle activity, and muscle fatigue.

In some embodiments, the method 900 further comprises collecting second biometric data at a sampling rate of at least 10 kilosamples per second using a second flexible transducer, wherein the first flexible transducer is worn in a first area of the person's body having a first rate of motion, the second flexible transducer is worn in a second area of the body having a second rate of motion, the second rate of motion being greater than the first rate of motion, and at least one of a data acquisition rate and a data transmission rate of the second flexible transducer is greater than a corresponding rate of the first flexible transducer.

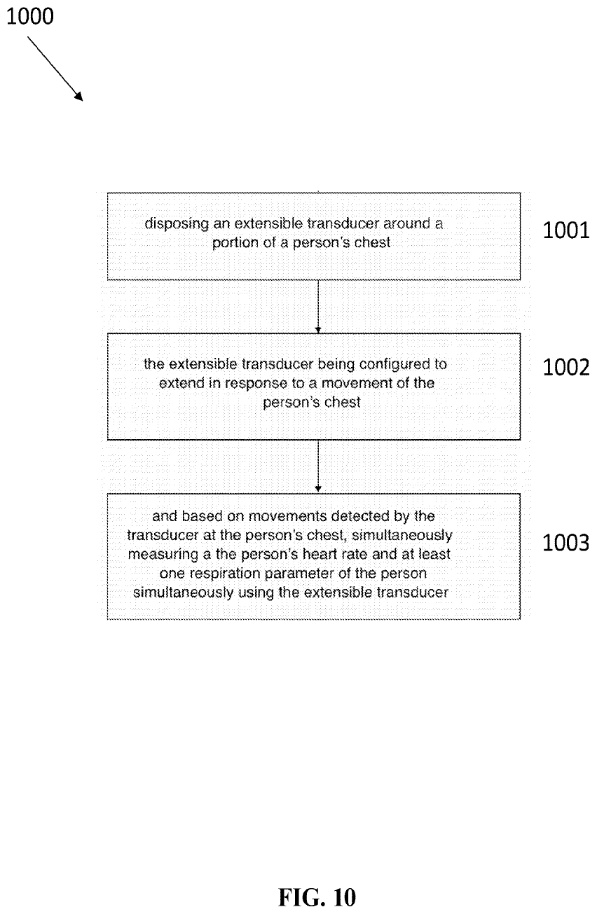

FIG. 10 shows a flowchart detailing an exemplary method 1000 for biometric data acquisition, according to an embodiment of the present disclosure. The method 1000 comprises disposing an extensible transducer around a portion of a person's chest (1001), the extensible transducer being configured to extend in response to movement of the person's chest (1002), and, based on movements detected by the transducer at the person's chest, simultaneously measuring a heartrate and at least one respiration parameter of the person using the extensible transducer (1003). In some embodiments, the extensible transducer comprises an optical fiber, wherein the optical fiber comprises a first end configured to receive light emitted by a light source and a second end configured to transmit light to a detector, wherein at least a portion of the optical fiber is deformable and has a propagation loss parameter configured to increase when the deformable section is deformed.

In some embodiments, the extensible transducer is configured to obtain data indicating the movement of the person's chest over a dynamic range from an extension of about 0% to an extension of about at least 15%. In some embodiments, the extensible transducer is configured to obtain data indicating the movement of the person's chest to an extension of about at least 25%. In some embodiments, the extensible transducer is configured to obtain data indicating the movement of the person's chest to an extension of about at least 50%. In some embodiments, over the dynamic range, the extensible transducer is configured to measure the movement of the person's chest to within one millimeter. In some embodiments, the extensible transducer is configured to measure the movement of the person's chest to within one-tenth of a millimeter. In some embodiments, the extensible transducer is configured to measure forces less than 1 N, and wherein the force is measureable to within 0.01 N. In some embodiments, the extensible transducer is configured to simultaneously measure both a heartrate and a respiration parameter of the person. In some embodiments, the extensible transducer is configured to measure strain in the range of about 100 μm to about 10 cm, wherein the strain is measurable to within 100 μm strain. In some embodiments, the biometric data is collected at a sampling rate of at least 100 kilosamples per second. In some embodiments, the biometric data is collected at a sampling rate of at least 200 kilosamples per second. In some embodiments, the biometric data indicating a movement of a person's body are measured at kHz frequencies.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

A1. A system for biometric data acquisition, the system comprising:
a first flexible transducer configured to be worn around or along a portion of a person's body, the first flexible transducer is configured to obtain biometric data indicating a movement of the person's body;
a processor; and
a transmitter,
wherein the system is configured to collect biometric data at a sampling rate of at least 10 kilosamples per second.

A2. The system of claim A1, wherein the first flexible transducer comprises:
an optical fiber, the optical fiber comprising:
a first end configured to receive light emitted by a light source;
a second end configured to transmit light to a detector; and
at least a portion of the optical fiber being deformable and having a propagation loss parameter, the propagation loss parameter being configured to increase when the deformable section is deformed.

A3. The system of any of claims A1-A2, the system further comprising a second flexible transducer, wherein:
the first flexible transducer is configured to be worn in a first area of the person's body having a first rate of motion;
the second flexible transducer is configured to be worn in a second area having a second rate of motion, the second rate of motion being greater than the first rate of motion; and
at least one of a data acquisition rate and a data transmission rate of the second flexible transducer is greater than a corresponding rate of the first flexible transducer.

A4. The system of any of claims A1-A3, wherein the system is configured to collect biometric data at a sampling rate of at least 100 kilosamples per second.

A5. The system of any of claims A1-A4, wherein the system is configured to collect biometric data at a sampling rate of at least 200 kilosamples per second.

A6. The system of any of claims A1-A5, wherein the biometric data comprises at least one of heartrate, chest excursion, tidal volume, minute volume, vital capacity, breathing rate, joint angle or position, joint velocity, joint acceleration, jerk, snap, joint stiffness, muscle activity, and muscle fatigue.

A7. The system of any of claims A1-A6, wherein a first end and a second end of the first flexible transducer are anchored in a wearable garment.

A8. The system of any of claims A1-A6, wherein the first end and the second end of the first flexible transducer are anchored in a wearable band.

A9. The system of claim A8, wherein the wearable band is an orthogonal band configured to be worn along a length of a person's joint.

A10. The system of claim A8, wherein the wearable band is a transverse or horizontal band configured to be worn circumferentially around a section of the person's body.

A11. The system of any of claims A1-A10, wherein the system is in the form of a modular pod capable of being coupled to and decoupled from a wearable garment or a wearable band.

B1. A method for biometric data acquisition, the method comprising:
collecting biometric data at a sampling rate of at least 10 kilosamples per second; and
transmitting a wireless signal comprising the biometric data to a receiver at a speed of at least 0.1 kbps;
wherein the step of collecting the biometric data is performed using a system comprising:
a first flexible transducer configured to be worn around or along a portion of a person's body, the first flexible transducer is configured to obtain biometric data indicating a movement of the person's body;
a microcontroller;
an analog-to-digital converter; and
a transmitter.

B2. The method of claim B1, wherein the first flexible transducer comprises:
an optical fiber, the optical fiber comprising:
a first end configured to receive light emitted by a light source;
a second end configured to transmit light to a detector; and
at least a portion of the optical fiber being deformable and having a propagation loss parameter, the propagation loss parameter being configured to increase when the deformable section is deformed.

B3. The method of any of claims B1-B2, the method further comprising:
collecting second biometric data at a sampling rate of at least 10 kilosamples per second using a second flexible transducer;
wherein:
the first flexible transducer is configured to be worn in a first area of the person's body having a first rate of motion;
the second flexible transducer is configured to be worn in a second area having a second rate of motion, the second rate of motion being greater than the first rate of motion; and
at least one of a data acquisition rate and a data transmission rate of the second flexible transducer is greater than a corresponding rate of the first flexible transducer.

B4. The method of any of claims B1-B3, wherein the biometric data is collected at a sampling rate of at least 100 kilosamples per second.

B5. The method of any of claims B1-B4, wherein the biometric data is collected at a sampling rate of at least 200 kilosamples per second.

B6. The method of any of claims B1-B5, wherein the biometric data comprises at least one of heartrate, chest excursion, tidal volume, minute volume, vital capacity, breathing rate, joint angle or position, joint velocity, joint acceleration, jerk, snap, joint stiffness, muscle activity, and muscle fatigue.

B7. The method of any of claims B1-B6, wherein a first end and a second end of the first flexible transducer are anchored in a wearable garment.

B8. The method of any of claims B1-B6, wherein the first end and the second end of the first flexible transducer are anchored in a wearable band.

B9. The method of claim B8, wherein the wearable band is an orthogonal band configured to be worn along a length of a person's joint.

B10. The method of claim B8, wherein the wearable band is horizontal or transverse band configured to be worn circumferentially around a section of the person's body.

B11. The method of any of claims B1-B10, wherein the first flexible transducer is coupled to a modular pod capable of being coupled to and decoupled from a wearable garment or a wearable band.

C1. A system for monitoring biometric data, the system comprising:
an extensible transducer configured to be worn around a person's chest, the extensible transducer being configured to extend in response to a movement of the person's chest;
wherein:
the extensible transducer is configured to obtain data indicating the movement of the person's chest over a range from an extension of 0% to an extension of at least 50%;
over the range of 0% to 50% extension, the extensible transducer is configured to measure the movement of the person's chest to within one millimeter; and
the extensible transducer is configured to simultaneously measure both a heart rate and a respiration parameter of the person.

C2. The system of claim C1, wherein the system is configured to measure forces less than 1 N.

C3. The system of claim C2, wherein the force is measurable to within 0.01 N.

C4. The system of any of claims C1-C4, wherein the system is configured to measure strain in at least a range of 100 μm to 10 cm.

C5. The system of claim C4, wherein the strain is measurable to within 100 μm strain.

C6. The system of any of claims C1-C5, wherein the heart rate is measured from a pulse beating, and wherein the at least one respiration parameter is one or more of chest excursion, tidal volume, minute volume, vital capacity, and breathing rate.

C7. The system of claim C6, wherein the heart rate is measured in beats per minute the breathing rate is measured in breaths per minute.

C8. The system of any of claims C1-C7, wherein the system is configured to collect biometric data at a sampling rate of at least 100 kilosamples per second.

C9. The system of any of claims C1-C8, wherein the extensible transducer comprises an optical fiber, the optical fiber comprising:
a first end configured to receive light emitted by a light source;
a second end configured to transmit light to a detector; and
a deformable section having a propagation loss parameter, the propagation loss parameter being configured to increase when the deformable section is deformed.

D1. A method for biometric data acquisition, the method comprising:
disposing an extensible transducer around a portion of a person's chest, the extensible transducer being configured to extend in response to a movement of the person's chest; and
based on movements detected by the transducer at the person's chest, simultaneously measuring a heart rate and at least one respiration parameter of the person using the extensible transducer;
wherein:
the extensible transducer is configured to obtain data indicating the movement of the person's chest over a range from an extension of 0% to an extension of at least 50%;
over the range of 0% to 50% extension, the extensible transducer is configured to measure the movement of the person's chest to within one millimeter.

D2. The method of claim D1, wherein the extensible transducer is configured to measure forces less than 1 N.

D3. The method of claim D2, wherein the force is measurable to within 0.01 N.

D4. The method of any of claims D1-D4, wherein the extensible transducer is configured to measure strain in at least a range of 100 μm to 10 cm.

D5. The method of claim D4, wherein the strain is measurable to within 100 μm strain.

D6. The method of any of claims D1-D5, wherein the heart rate is measured from a pulse beating, and the at least one respiration parameter is one or more of chest excursion, tidal volume, minute volume, vital capacity, and breathing rate.

D7. The method of claim D6, wherein the heart rate is measured in beats per minute and the breathing rate is measured in breaths per minute.

D8. The method of any of claims D1-D7, wherein the extensible transducer is configured to collect biometric data at a sampling rate of at least 100 kilosamples per second.

D9. The method of any of claims D1-D8, wherein the extensible transducer comprises an optical fiber, the optical fiber comprising:
a first end configured to receive light emitted by a light source;
a second end configured to transmit light to a detector; and
a deformable section having a propagation loss parameter, the propagation loss parameter being configured to increase when the deformable section is deformed.
